(19) United States
(12) Patent Application Publication
(10) Pub. No.: US 2012/0136243 A1
(43) Pub. Date: May 31, 2012

(54) METHOD FOR CALCULATING PERFUSION DATA

(76) Inventors: Jan Boese, Eckental (DE); Andreas Fieselmann, Erlangen (DE); Klaus Klingenbeck, Aufsess (DE); Markus Kowarschik, Langenzenn (DE)

(21) Appl. No.: 13/302,170

(22) Filed: Nov. 22, 2011

(30) Foreign Application Priority Data
Nov. 26, 2010 (DE) 102010062030.0

(51) Int. Cl. A61B 5/05

(52) U.S. Cl. 600/425

(57) ABSTRACT

A method for calculating perfusion data, such as blood volume or blood flow from 2-D angiography data or DSA sequences, is proposed. An angiography scene is recorded using specific acquisition parameters to generate the 2-D angiography data or DSA sequences with administration of contrast agent based on a multiplicity of individual angiography images. A region of interest is defined suitable for comparison purposes. The volume segments are defined by the region of interest. The time/contrast curve is determined in the volume segments. Perfusion data for calculating the relative perfusion data is ascertained. The perfusion data is compared and the relative perfusion data is calculated. The calculated relative perfusion data is not specified in terms of absolute physical quantities, but is provided simply as ratios, such as left/right or before/after.

### CROSS REFERENCE TO RELATED APPLICATIONS

This application claims priority of German application No. 10 2010 062 030.0 filed Nov. 26, 2010, which is incorporated by reference herein in its entirety.

### FIELD OF THE INVENTION

The invention relates to a method for calculating perfusion data, such as blood volume or blood flow for example, from 2-D angiography data or DSA sequences.

### BACKGROUND OF THE INVENTION

The blood supply can be compromised inter alia by stenoses in blood vessels. These can either be treated through medication or by means of an angioplasty—with or without stent—or alternatively be circumvented by means of a bypass, of coronaries for example.

The success of these treatments is usually demonstrated by the diameter of the vessel both before and after a treatment or determined according to the subjectively optical distribution of the contrast agent in an image of the vessel acquired for example by means of DSA (Digital Subtraction Angiography). However, the treatment with medication in particular has no effect on the vessel diameter. In the case of perfusion deficits caused by spasms it is nonetheless in fact true that the administration of drugs (e.g. nimodipine) leads to a dilation of the vessel.

A typical X-ray system by means of which DSA sequences of the aforesaid type can be produced is shown for example in FIG. 1, which depicts a monoplane X-ray system having a C-arm 2 which is mounted on a pedestal 1 in the form of a six-axis industrial or articulated-arm robot and at the ends of which are attached an X-ray radiation source, for example an X-ray emitter 3 comprising X-ray tube and collimator, and an X-ray image detector 4 as image acquisition unit.

The C-arm 2 can be adjusted arbitrarily in space by means of the articulated-arm robot known for example from U.S. Pat. No. 7,500,784 B2, which robot preferably has six axes of rotation and therefore six degrees of freedom, for example in that it is rotated about a center of rotation between the X-ray emitter 3 and the X-ray image detector 4. The angiographic X-ray system 1 to 4 according to the invention is rotatable in particular about centers of rotation and axes of rotation in the C-arm plane of the X-ray image detector 4, preferably about the center point of the X-ray image detector 4 and about axes of rotation intersecting the center point of the X-ray image detector 4.

The known articulated-arm robot has a base frame which is permanently installed on a floor for example. Attached thereto is a carousel which is rotatable about a first axis of rotation. Mounted on the carousel so as to be pivotable about a second axis of rotation is a robot rocker arm to which is attached a robot arm which is rotatable about a third axis of rotation. Mounted at the end of the robot arm is a robot hand which is rotatable about a fourth axis of rotation. The robot hand has a retaining element for the C-arm 2, said retaining element being pivotable about a fifth axis of rotation and rotatable about a sixth axis of rotation running perpendicular thereto.

The X-ray diagnostic apparatus is not dependent for its implementation on the industrial robot. Conventional C-arm devices having a standard ceiling- or floor-mounted retaining fixture for the C-arm 2 can also be used. Instead of the C-arm 2 shown by way of example, the angiographic X-ray system can also have separate ceiling- and/or floor-mounted retaining fixtures for the X-ray emitter 3 and the X-ray image detector 4 which are rigidly coupled electronically, for example.

The X-ray image detector 4 can be a rectangular or square, flat semiconductor detector which is preferably produced from amorphous silicon (a-Si). Integrating and possibly counting CMOS detectors can also be used, however.

A patient 6 that is to be examined is located as the examination subject in the beam path of the X-ray emitter 3 on a tabletop 5 of a patient positioning table. Connected to the X-ray diagnostic apparatus is a system control unit 7 having an imaging system 8 which receives and processes the image signals from the X-ray image detector 4 (control elements are not shown, for example). The X-ray images can then be studied on displays of a traffic-light monitor array 9.

Numerous diagnostic and therapeutic applications require information in relation to tissue perfusion. What is understood by this general term is quantitative information in respect of the blood flow through tissue regions such as, for example, tumors in the oncology environment or infarction-threatened cerebral areas in the neurology domain. Key perfusion parameters include the blood volume (static, typically specified in ml/100 g) and the blood flow (dynamic, typically specified in ml/100 g/min).

Perfusion measurements are established methods in computed tomography (CT), in magnetic resonance tomography (MRT) and in nuclear medicine. Ultrasound technology also permits conclusions in relation to perfusions to be reached to a limited degree.

To date, however, there still exists no practical prior art approach to extracting perfusion data such as blood volume and blood flow from 2-D angiography data or DSA sequences.

Theoretical preliminary work is described in “Estimating perfusion using X-ray angiography” by Hrvoje Bogunovic and Sven Loncaric, Proc. IEEE ISPA, 2005, pages 147 to 150. However, this approach as described in Bogunovic et al. has the disadvantage that the perfusion measurements are dependent on the injection profile of the contrast agent bolus. Moreover the approach according to Bogunovic et al. yields only qualitative results, since proportionality constants are ignored.

In computed tomography, which constitutes an imaging method in 3-D, there exist various physical models and approaches which serve as a basis for the calculation of CT perfusion data and some of these are also already available as products. These models serve as a starting point for present approaches based on 2-D image series.

### SUMMARY OF THE INVENTION

The invention is based on the object of embodying a method of the type cited in the introduction in such a way that relative perfusion data in respect of blood volume and blood flow can be extracted in a simple manner from 2-D angiography data or from 2-D DSA sequences.

The object is achieved according to the invention by the features disclosed in the independent claim. Advantageous embodiments are set forth in the dependent claims.

The object is achieved according to the invention by means of the following steps:
S1) recording at least one angiography scene using specific acquisition parameters in order to generate the 2-D angiography data or DSA sequences with administration of contrast agent based on a multiplicity of individual angiography images,
S2) defining a region of interest suitable for comparison purposes,
S3) calculating the volume segments defined by the region of interest,
S4) determining the time/contrast curve in the volume segments,
S5) ascertaining perfusion data for the purpose of calculating the relative perfusion data,
S6) comparing the perfusion data,
S7) calculating the relative perfusion data, and
S8) rendering the calculated relative perfusion data.

The invention therefore relates to a method for determining relative perfusion data such as blood volume and blood flow for example. The term “relative” refers to the fact that the perfusion data (blood volume and blood flow) calculated by means of the method steps according to the invention is not specified in terms of absolute physical quantities, but is provided simply as ratios (left/right or before/after (pre-/post-treatment)).

By defining a region of interest suitable for comparison purposes it is possible to arrive at conclusions about the mass ratio of the contrast agent associated with respective time instants (and hence of the blood, provided an ideal mixture of blood and contrast agent is assumed) within the volume segments defined by the region of interest, such that the relative blood volume and/or the relative blood flow can be calculated.

However, if absolute perfusion data, for example from previous CT perfusion examinations (CTP examinations), is available, then approximations for absolute perfusion data can again be derived from the relative perfusion data.

It has proven advantageous if an angulation of the angiography system is chosen which has the fewest possible interfering overlays of the tissue region that is to be examined by blood vessels lying spatially in front of or behind said tissue region. The greater the number of such overlays occurring, the more inaccurate will be the estimation of the relative perfusion parameters.

According to the invention the perfusion data according to step S5) can be the blood volume and/or the blood flow.

The comparison according to step S6) can advantageously be carried out at two time instants in the before/after comparison and/or at two locations in the left/right comparison.

It has proven advantageous, for the purpose of the comparison according to step S6), to form the ratio of slopes, intensities, areas at a specific time instant and/or of the maxima of the intensities of the time/contrast curves.

According to the invention changes to exposure parameters due to regulating actions of a system control unit can be calculated out of the image sequences by means of an imaging system. Said changes to exposure parameters can result for example from an automatic dose regulation by the angiography system.

During the definition of a region of interest (ROI) according to step S2) for the purpose of a before/after comparison at the respective time instant, the acquisition parameters can advantageously be kept constant, for example in the case of tumor embolizations, wherein according to the invention the acquisition parameters that are kept constant can be the angulation of the C-arm, the zoom factor used and the injection protocol.

During the definition of a region of interest according to step S2) for the purpose of left/right comparisons it is possible according to the invention to choose an injection protocol which prefers no half of the body per se. Typically a stationary state is necessary for determining the blood volume in a tissue region.

According to the invention the left/right comparison can be carried out in the brain or in paired organs, such as the kidneys for example.

It has proven advantageous if, upon presentation of absolute perfusion data from previous CT perfusion examinations, approximations for absolute perfusion data are again derived from the relative perfusion data.

### BRIEF DESCRIPTION OF THE DRAWINGS

The invention is explained in more detail below with reference to exemplary embodiments illustrated in the drawing, in which:

FIG. 1 shows a known biplane C-arm X-ray system for neuroradiology,

FIG. 2 shows a 3-D region which is implicitly defined by means of a chosen 2-D region of interest,

FIG. 3 shows a first time/contrast curve before the treatment,

FIG. 4 shows a second time/contrast curve after the treatment,

FIG. 5 shows a first simplified time/contrast curve,

FIG. 6 shows a second simplified time/contrast curve,

FIG. 7 shows a first time/contrast curve before the treatment,

FIG. 8 shows a second time/contrast curve after the treatment,

FIG. 9 shows a simplified model of a first time/contrast curve, and

FIG. 10 shows a simplified model of a second time/contrast curve.

### DETAILED DESCRIPTION OF THE INVENTION

The following terms should first be clarified at the outset:

Region of Interest (ROI):

The invention is based on the appropriate definition of regions of interest (ROIs), as will be explained briefly with reference to FIG. 2. In a 2-D X-ray or angiography image 11 containing blood vessels 12 for example, what is understood quite generally by an ROI is a user-defined section of the angiography image 11. As a result of the preceding subtraction of the mask image in the case of DSA sequences the sum of the grayscale values of all pixels lying within the ROI in each individual image of the sequence is directly proportional to the mass of the contrast agent that is contained in the 3-D volume segment V_{ROI} defined by the ROI. Since no depth information at all is in fact contained in a 2-D X-ray image 11, present approaches consequently always acquire the 3-D volume segment V_{ROI} which is “excised” by the ROI defined in 2-D and which extends over the entire object depth.

It is important for the application of the approaches proposed here that an angulation of the angiography system is chosen which has the fewest possible overlays of the tissue region that is to be examined by blood vessels 12 lying spatially in front of or behind said tissue region. The greater the number of such overlays occurring, the more inaccurate will be the estimation of the relative perfusion parameters.

Relative Blood Volume from 2-D Angiography Data:

Defining suitable ROIs—either for the purpose of before/after comparisons (e.g. in the case of tumor embolizations) or for the purpose of left/right comparisons either in the brain or in paired organs such as the kidneys for example—enables results to be computed in relation to the mass ratio of the contrast agent associated with the respective time instant (in left/right comparisons) or with the respective two time instants (in before/after comparisons) (and hence of the blood, provided an ideal mixture of blood and contrast agent is assumed) within the volume segments V_{ROI} defined by the ROIs.

Meaningful determinations of the relative blood volume naturally demand here that in the case of before/after comparisons the acquisition parameters, such as in particular the angulation of the C-arm and the zoom factor used for example, and the injection protocol remain constant. Any changes to the exposure parameters resulting from the automatic dose regulation by the angiography system must be calculated out of the image sequences accordingly in order to allow a meaningful comparison. In the case of left/right comparisons it is of course likewise necessary to choose a suitable injection protocol which prefers no half of the body per se. Typically a stationary state is required for determining the blood volume in a tissue region.

According to the theory (see equation (5) in Konstas et al.) the blood volume V in a volume segment can in fact also be calculated from dynamic data as follows:

$$V ∝ \frac{{\int }_{0}^{T} {C}_{tissue} ( t ) d t}{{\int }_{0}^{T} {C}_{artery} ( t ) d t} . \qquad ( 1 )$$

Cₜᵢₛₛᵤₑ(t) denotes the average contrast agent concentration in the tissue region under examination, while C_{artery}(t) denotes the sum of the average contrast agent concentrations in the supplying arteries. In this case the upper integration limit T should be suitably chosen to enable the transported contrast agent bolus to be recorded completely. However, the integration should only include the time in which the contrast agent bolus undertakes a first pass through the tissue so that distortions of the values caused by recirculation of the bolus are avoided.

In before/after comparisons with constant injection protocol and constant acquisition parameters as well as in left/right comparisons with appropriately chosen injection protocol it may be assumed for simplicity that the arterial input left and right or, as the case may be, before and after is consistent, such that in the case of before/after comparisons the relation

$$\frac{{V}_{after}}{{V}_{before}} = \frac{{\int }_{0}^{T} {C}_{tissue , after} ( t ) d t}{{\int }_{0}^{T} {C}_{tissue , before} ( t ) d t} \qquad ( 2 t )$$

is obtained and in the case of left/right comparisons the analog relation

$$\frac{{V}_{left}}{{V}_{right}} = \frac{{\int }_{0}^{T} {C}_{tissue , left} ( t ) d t}{{\int }_{0}^{T} {C}_{tissue , right} ( t ) d t} \qquad ( 2 o )$$

is obtained. It should be noted that a change in the blood flow (specified in ml/100 g/min) in the case of before/after comparisons or a different blood flow left/right in the case of left/right comparisons has no relevance, since the flow has already been eliminated in the course of the derivation of equation (1). Equation (1) henceforth includes only the time-dependent contrast agent concentrations.

Taking into account that the concentration of the contrast agent is proportional to the mass of the contrast agent (concentration=mass/volume), and assuming that the volume segments being examined are present with at least approximately the same size (both in before/after and in left/right comparisons), the proportionality constants (1/volume) are omitted in the above formulae and the corresponding relative blood volumes can be expressed by means of the contrast agent masses. As already mentioned further above, the contrast agent masses are in turn proportional to the sums of the grayscale values of all pixels lying within the ROIs (in each individual image of the sequence).

Accordingly the relative blood volumes can be determined as follows:

$$\frac{{V}_{after}}{{V}_{before}} = \frac{{\int }_{0}^{T} {m}_{after} ( t ) d t}{{\int }_{0}^{T} {m}_{before} ( t ) d t} \qquad ( 3 t )$$

and analogously thereto

$$\frac{{V}_{left}}{{V}_{right}} = \frac{{\int }_{0}^{T} {m}_{left} ( t ) d t}{{\int }_{0}^{T} {m}_{right} ( t ) d t} . \qquad ( 3 o )$$

In the two previous formulae, therefore, the time integrals are placed over the ROI-specific time/contrast curves in the numerator and in the denominator in each case.

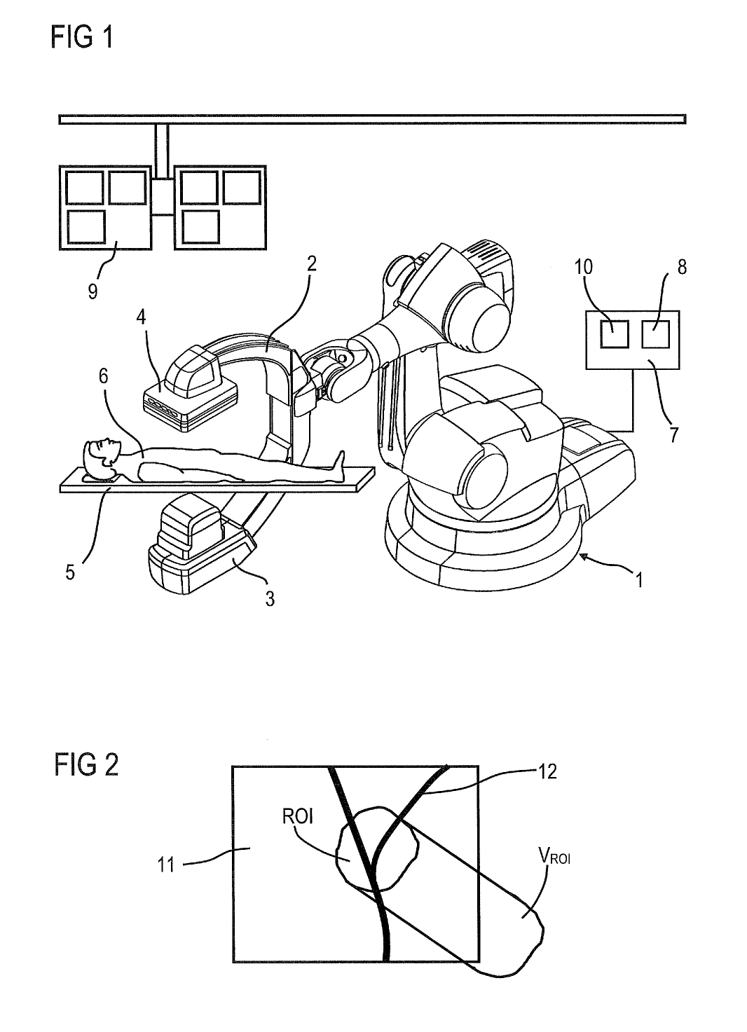

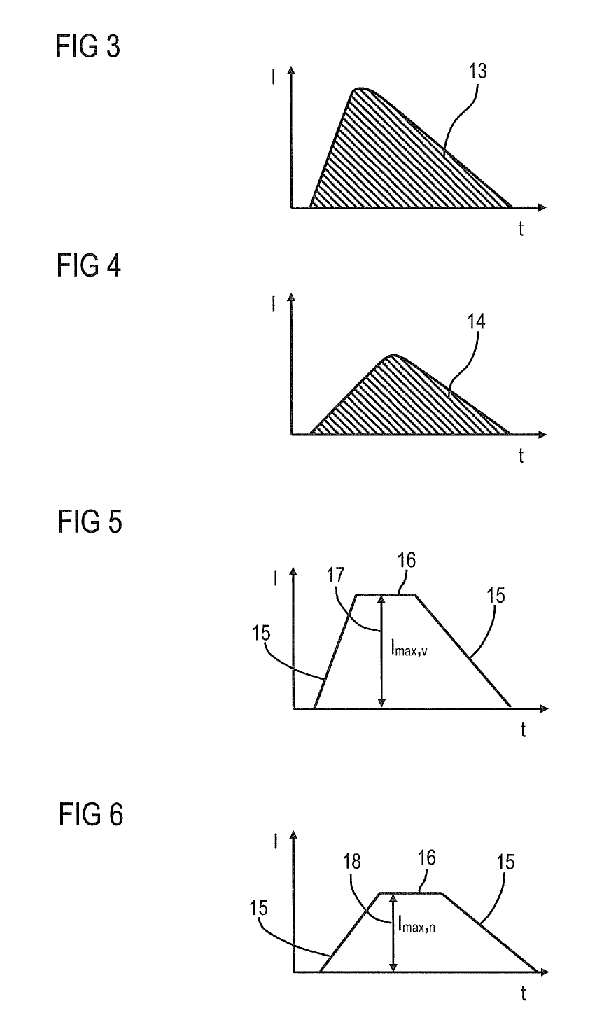

The general case of the calculation of the change in relative blood volume is explained in more detail with reference to FIGS. 3 to 6. For the purpose of determining relative perfusion data according to the invention a perfusion measurement device 10 is provided in the system control unit 7, as shown in FIG. 1. As output of the calculated perfusion data this also effects an insertion for example as a numeric value characteristic of the ROI into the image on a display of the traffic-light monitor array 9.

FIG. 3 shows a first time/contrast curve 13 I/t before the treatment and FIG. 4 shows a second time/contrast curve 14 I/t after the treatment. The area AUC (area under the curve) under the overall curves 13 and 14 is formed by the time integrals. Their ratio expresses a change in relative blood volume. In order to calculate the change in relative blood volume the areas under the overall curves 13 and 14 can now be put into the ratio AUC_{after}/AUC_{before}.

For simplicity the calculation of the integrals according to the examples explained with reference to FIGS. 3 and 4 can be dispensed with here and in each case the maximum of the associated time/contrast curve can be used instead, as is shown with reference to FIGS. 5 and 6 (in this regard see also FIG. 2 in Konstas et al. “Theoretic Basis and Technical Implementations of CT Perfusion in Acute Ischemic Stroke, Part 1: Theoretic Basis”, AJNR Am. J. Neuroradiol. 30, 2009, pages 662 to 668). This simplification is based on the assumption that there are plateau-like maxima of the time/contrast curves at which a saturated state of the contrast agent concentration can be assumed. The advantage of this simplification consists in the fact that it is not necessary to integrate over a relatively long time period and therefore overlay effects caused by the contrast agent flow in draining veins, which could of course also be visible in the projection image, are avoided. However, this simplifying estimation of the relative blood volume requires a greater amount of contrast agent to be administered in order to achieve the stationary state, which is not always desirable or feasible.

According to the invention the calculation can now be simplified in that, as shown in FIGS. 5 and 6, the slopes 15 and the maxima 16 of the first simplified time/contrast curve before the treatment and the second simplified time/contrast curve after the treatment are assumed to be straight lines. The maximum intensity 17 I_{max,v} before the treatment and the maximum intensity 18 I_{max,n} after the treatment can then be ascertained in a simple manner.

In order to calculate the simplified change in relative blood volume the two maximum intensities are now put into the ratio I_{max,after}/I_{max,before}.

### Example

In the case of a tumor embolization the tumor can be characterized in the two DSA sequences (pre- and post-treatment (before/after)) by means of an ROI in each case and then the ratio of the time integrals over the two time/contrast curves determined. According to the above consideration their quotient represents the ratio of the blood volumes before and after the intervention. Ideally, no more contrast agent at all accumulates in the tumor after the embolization, thus yielding the ratio V_{after}/V_{before}˜0 as result. As already mentioned, a suitable angulation must be chosen for an examination of said type to ensure that no large blood vessels run through the volume segment defined by means of the ROI, since these would distort the result.

Relative Blood Flow from 2-D Angiography Data:

The relative blood flow can be determined in a comparable way to the determining of the relative blood volume. In this case the so-called “maximum slope method” can be used, see equation (10) in Konstas et al. In spite of simplifying assumptions this method is also employed in CT for the purpose of measuring the blood flow. This method provides a simple computing rule for determining the flow F which is assumed as constant over time:

$${[ \frac{d m ( t )}{d t} ]}_{max} = F \cdot {[ {C}_{artery} ( t ) ]}_{max}$$

Here, m(t) denotes the mass of contrast agent contained in the tissue volume under examination at the time instant t, and C_{artery}(t) denotes the contrast agent concentration in the supplying artery at the time instant t. For the sake of simplicity it is assumed that no venous outflow takes place during the examination time period and that precisely one artery supplies the examined tissue volume. According to this relation the flow F can therefore be determined by dividing the maximum rise of the mass of contrast agent in the tissue by the maximum contrast agent concentration in the supplying artery.

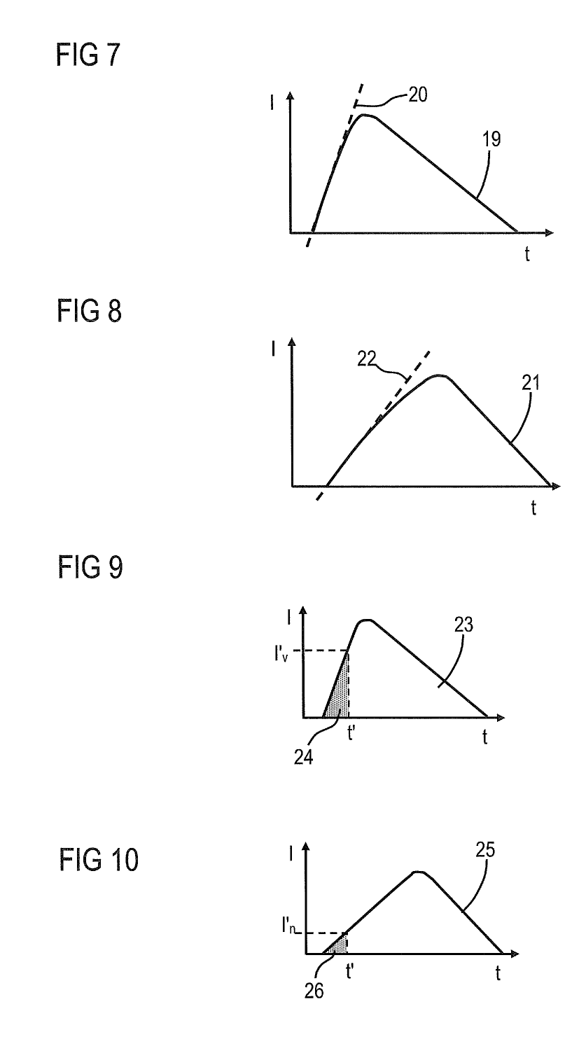

The general case of the calculation of the change in relative blood flow is explained in more detail with reference to FIGS. 7 and 8. In this case FIG. 7 shows a first time/contrast curve 19 before the treatment. A first maximum slope 20 is applied to the ascending branch of said first time/contrast curve 19. FIG. 8 shows a second time/contrast curve 21 after the treatment, to the ascending branch of which a second maximum slope 22 is applied.

As also in the case of the determining of the relative blood volume from 2-D angiography data, suitable ROIs should be defined in 2-D at a suitable angulation of the C-arm, which ROIs then again characterize 3-D volume segments that extend over the entire object depth. On the assumption that the arterial inflow left/right or before/after is the same, the relative blood flow can be approximated as follows in the case of left/right comparisons according to the formula

$$\frac{{F}_{left}}{{F}_{right}} = \frac{{[ \frac{d {m}_{left} ( t )}{d t} ]}_{max}}{{[ \frac{d {m}_{right} ( t )}{d t} ]}_{max}} \qquad ( 4 o )$$

and in the case of before/after comparisons according to the formula

$$\frac{{F}_{after}}{{F}_{before}} = \frac{{[ \frac{d {m}_{after} ( t )}{d t} ]}_{max}}{{[ \frac{d {m}_{before} ( t )}{d t} ]}_{max}} . \qquad ( 4 t )$$

This means that—owing to the direct proportionality of contrast agent mass and the attenuation along the X-ray beams—the quotients from the maximum slopes 20 and 22 of the time/contrast curves 19 and 21 must be formed in order to obtain the corresponding estimations of the relative blood flow.

Thus, as was already the case in the determining of the relative blood volumes, the “trick” consists in determining the relative flows (left/right and/or after/before), since then the proportionality constants, which are not known due to the absence of depth information, are omitted from the formation of the quotients.

The simplified case of the calculation of the change in relative blood flow is explained in more detail below with reference to FIGS. 9 and 10. Instead of the maximum slopes 20 and 22 that were explained with reference to FIGS. 7 and 8, alternative parameters for determining blood flow can also be chosen if a specific model of the time/contrast curves I/t is assumed for simplicity.

In this simplified model it is assumed that a first time/contrast curve 23 rises linearly until saturation is reached, as revealed in FIGS. 9 and 10. It is easy to show that the slope 24 of the first simplified time/contrast curve 23 in this rise phase is proportional to two other parameters. The first parameter is the intensity value I′ᵥ at a time instant t′, which must chosen such that it lies before the maximum contrast is reached. The second parameter is the first integral 25 (area under the curve (AUC)) of the first time/contrast curve 23 up to the time instant t′.

The same also applies to the after case shown in FIG. 10, in which a linear rise of a second simplified time/contrast curve 26 until saturation is reached is likewise assumed. Here too it holds that the slope 27 of the second simplified time/contrast curve 26 in this rise phase is proportional to the intensity value I′ₙ at the time instant t′. The second integral 28 of the second simplified time/contrast curve 26 up to the time instant t′ can also be drawn upon again here as the second parameter.

Since these two parameters are proportional to the maximum slope, they can likewise be used for calculating the relative flow by formation of the quotients of the values before and after a treatment (or, of course, also referred to a left/right comparison).

The change in relative blood flow can therefore be calculated in a simplified manner as follows:

I′_{after}/I′_{before}=AUC_{after}/AUC_{before}≈m_{after}/m_{before}

where m is the maximum slope and AUC is the area under the time/contrast curve I/t.

It is important to bear in mind that this simplifying assumption of a linear rise together with the associated simplified estimation of the relative blood flow has nothing to do with the above-explained assumption of a stationary state which leads to a simplified estimation of the relative blood volume.

The invention relates to an imaging method for calculating and deriving relative perfusion data, such as blood volume or blood flow for example, from 2-D angiography data, for example 2-D DSA sequences. To clarify: Per se this perfusion data represents absolute values (e.g. where CT perfusion is concerned). In the case of a 2-D image series this restriction to relative perfusion data must be applied, since no depth information at all is available. By waiving the requirement for absolute data and considering relative data by quotient formation it is possible to dispense with the depth information, which, of course, is not contained in the 2-D image sequences. Put more precisely, this dispenses with knowledge of the proportionality constant which relates the mass of the contrast agent along an X-ray beam to the concentration of the contrast agent along said X-ray beam.

1. A method for calculating a relative perfusion data, comprising:
recording an angiography image of a patient by an angiography system using specific acquisition parameters with administration of a contrast agent;
defining a region of interest on the angiography image that is suitable for comparison;
calculating a volume segment defined by the region of interest;
determining a time/contrast curve in the volume segment;
ascertaining perfusion data from the time/contrast curve;
comparing the perfusion data;
calculating the relative perfusion data from the comparison; and
outputting the calculated relative perfusion data.

2. The method as claimed in claim 1, wherein the angiography image is recorded in an angulation having the fewest interfering overlay of a tissue region with blood vessels lying spatially in front of or behind the tissue region.

3. The method as claimed in claim 1, wherein the perfusion data comprises blood volume and/or blood flow.

4. The method as claimed in claim 1, wherein ratio of slopes, intensities, areas at a specific time instant, and/or maxima of the intensities of the time/contrast curve is calculated for comparing the perfusion data.

5. The method as claimed in claim 1, wherein exposure parameters are calculated from the angiography image according to regulating actions of the angiography system.

6. The method as claimed in claim 1, wherein the perfusion data is compared at two time instants that are before and after a respective time instance.

7. The method as claimed in claim 6, wherein the acquisition parameters are kept constant at the two time instants.

8. The method as claimed in claim 7, wherein the acquisition parameters comprise angulation of the angiography system, zoom factor, and injection protocol.

9. The method as claimed in claim 1, wherein the perfusion data is compared at two locations that are left and right of a respective location.

10. The method as claimed in claim 9, wherein an injection protocol is no half of the patient for the left/right comparison.

11. The method as claimed in claim 9, wherein the left/right comparison is carried out in a brain or in paired organs of the patient.

12. The method as claimed in claim 11, wherein the paired organs comprise kidneys of the patient.

13. The method as claimed in claim 1, wherein an approximation for absolute perfusion data is derived from the relative perfusion data after presentation of the absolute perfusion data from a previous CT perfusion examination.
